(19) United States
(12) Patent Application Publication
(10) Pub. No.: US 2011/0033482 A1
(43) Pub. Date: Feb. 10, 2011

(54) HER3 AS A DETERMINANT FOR THE PROGNOSIS OF MELANOMA

(75) Inventors: Axel Ullrich, Munich (DE); Pjotr Knyazev, Stockdorf (DE); Markus Reschke, Munich (DE)

Correspondence Address: MEYERTONS, HOOD, KIVLIN, KOWERT & GOETZEL, P.C., P.O. BOX 398, AUSTIN, TX, 78767-0398, US

(73) Assignee: Max-Planck-Gesellschaft zur Förderung der Wissenschaften e. V.

(21) Appl. No.: 12/863,954

(22) PCT Filed: Jun. 26, 2009
(86) PCT No.: PCT/EP09/04648

§ 371 Date: Oct. 18, 2010

(30) Foreign Application Priority Data
Jun. 27, 2008 (EP) 08011736.9

(51) Int. Cl. A61K 39/395; C07K 16/18; C07H 21/02; A61K 31/713; A61P 35/00; C12Q 1/02

(52) U.S. Cl. 424/172.1; 530/387.1; 536/24.5; 514/44 A; 435/29

(57) ABSTRACT

Disclosed are inhibitors of HER3 for the treatment of melanoma, in particular melanoma metastases and/or refractory melanoma, pharmaceutical compositions comprising such an inhibitor and a method for the diagnosis or prognosis of melanoma.

### BACKGROUND OF THE INVENTION

1. Field of the Invention

The present invention relates to an inhibitor of HER3 for the treatment of melanoma, in particular melanoma metastases and/or refractory melanoma, pharmaceutical compositions comprising such an inhibitor and a method for the diagnosis or prognosis of melanoma.

2. Description of the Relevant Art

Melanoma is a common type of skin cancer, which develops from the malignant transformation of melanocytes, and accounts for 80% of deaths arising from skin cancer. The underlying molecular mechanisms of melanocyte transformation have been studied extensively in the past.

The human EGF receptor (HER) family of receptor tyrosine kinases regulates a large variety of biological processes including cell proliferation, -migration, -invasion and -survival. The family consists of four members: EGFR (HER1), HER2 (neu or ErbB2), HER3 (ErbB3) and HER4 (ErbB4). To date, eleven ligands have been reported including epidermal growth factor (EGF), heparin-binding EGF-like growth factor (HB-EGF), transforming growth factor α (TGFα), amphiregulin (AR), epiregulin, betacellulin and the heregulins. These ligands bind directly to their cognate receptors, which leads to the formation of receptor homo- or heterodimers that trigger the activation of multiple signaling pathways. Dysregulation of members of the HER-family either by activating mutations, receptor over expression or aberrant ligand release leads to the development of a variety of human tumors. HER3 is over expressed in breast-, ovarian- and lung cancer and this genetic feature has been correlated with poor prognosis. Upon activation by heregulins, HER3 dimerizes with HER2 and EGFR to form potent oncogenic receptor heterodimers. Within this complex, HER3 preferentially recruits PI3 kinase to its cytoplasmic docking sites thereby regulating cell proliferation and -survival. So far it was assumed that HER3 is kinase-inactive due to apparently aberrant sequence characteristics in its kinase domain and that it requires heterodimerization with a kinase-intact member of the HER-family in order to initiate signaling events. Consistent with this, it was shown that HER2 requires HER3 to drive breast tumor cell proliferation. However, recent findings of showed that HER3 is able to phosphorylate Pyk2 which results in the activation of the MAPK pathway in human glioma cells. Furthermore, monoclonal antibodies specific for HER3 can inhibit the proliferation and migration of cancer cell lines. Interestingly, it was shown recently that cancer cells escape HER-family inhibitor therapy by up-regulation of HER3 signaling and that HER3 inhibition abrogates HER2-driven tamoxifen resistance in breast cancer cells. Moreover, resistance to Gefitinib (Iressa) therapy, an EGFR small molecule inhibitor, was shown to be connected to HER3 signal activation.

The discovery of animal oncogenes that are derived from genes encoding receptor tyrosine kinases has led over the past twenty years to the development of several targeted therapeutics with the HER2 monoclonal antibody Trastuzumab being the first in clinical application for the treatment of metastatic breast carcinoma with HER2 gene amplification. However, in spite of further advances in the development of side effect-poor therapies for major malignancies such as breast cancer, there is still a great unmet need for better, more effective therapies for other cancer types. Melanoma is a highly aggressive skin cancer and current therapies only show limited efficacy in patients with late stage disease. So far it is known that the Ras-Raf-MAPK and the PI3K-AKT pathways are frequently activated in malignant melanoma and that this contributes to tumor progression.

To date no drugs are available that significantly prolong patient survival once melanoma progresses to the metastatic state. Thus, there is an urgent need for novel therapeutic agents and prognostic markers in the treatment of melanoma patients, in particular melanoma patients showing melanoma metastases.

### SUMMARY OF THE INVENTION

A therapeutic agent for the treatment of melanoma metastases and/or refractory melanoma comprises an inhibitor of HER3. The inhibitor of HER3, in an embodiment, is an inhibitor of HER3 expression, which preferably acts by down-regulation of HER3. In an embodiment, the inhibitor is an inhibitor of Heregulin-induced HER3 activation. In an embodiment, an inhibitor of HER3 includes nucleic acids, in particular siRNA, antisense oligonucleotides or ribozymes, peptidic compounds, in particular antibodies or antibody fragments and small organic molecules and combinations thereof.

The inhibitor may, in some embodiments, be an inhibitor of melanoma cell proliferation, migration and/or invasion. The refractory melanoma may be primary melanoma or metastases. Melanoma metastases are derived from or the refractory melanoma is selected from the group consisting of malignant melanoma, clear cell sarcoma, mucosal melanoma and/or uveal melanoma.

In an embodiment, the inhibitor of HER3 is an enhancer of apoptosis. The inhibitor of HER3 may sensitize the melanoma metastases and/or the refractory melanoma for treatment with a further agent, preferably a chemotherapeutic agent or radiation therapy.

In an embodiment, a pharmaceutical composition includes an inhibitor of HER3. The pharmaceutical composition may be administered in combination with further chemotherapeutic and/or immunotherapeutic agents and/or before, during and/or after surgical removal of the melanoma and/or radiation therapy. In an embodiment, the further agent is Dacarbazine, Herceptin, Omnitarg, Lapatinib, Temozolomide or combinations thereof. The pharmaceutical composition may be used for the treatment of patients expressing elevated levels of HER3.

In an embodiment an in vitro method for the diagnosis and/or prognosis of melanoma, preferably melanoma metastases, includes: (a) detecting HER3 levels in cells to be analyzed and; (b) correlating the detected HER3 levels with proliferation, tumor progression and/or patient survival and optionally; (c) administering an inhibitor according to any of the claims 1-9 or a pharmaceutical composition according to any of the claims 10-14. The detection of up-regulated HER3 levels indicate melanoma.

### BRIEF DESCRIPTION OF THE DRAWINGS

Advantages of the present invention will become apparent to those skilled in the art with the benefit of the following detailed description of embodiments and upon reference to the accompanying drawings in which:

FIGS. 1A-1D depict immunohistochemical staining of HER3 in primary melanoma and metastases;

FIG. 1E depicts a graph of HER3 expression frequencies in primary melanoma and metastases;

FIG. 1F depicts the absence of HER3 expression in primary melanocytes;

FIG. 2A depicts the increase of HER3 expression during melanoma progression;

FIG. 2B depicts a Kaplan-Meier analysis of tumor-specific survival according to HER3 expression levels;

FIG. 3A depicts Western blots for HER3 and Tubulin showing HER3 knock-down in Colo 829 and MM-358 melanoma cells;

FIG. 3B depicts the effect of HER3 knock-down on proliferation of Colo 829 and MM-358 melanoma cells;

FIG. 3C depicts the effect of HER3 knock-down on AKT activity upon heregulin β1 stimulation and shows how HER3 knock-down induces a growth arrest in Colo 829 and MM-358 melanoma cells;

FIG. 4A depicts the effect of HER3 knock-down on the migration of Colo 829, MM-358 and Mel Gerlach melanoma cells;

FIG. 4B depicts the effect HER3 knock-down on the invasion of Colo 829 and Mel Gerlach melanoma cells;

FIG. 5A depicts the effects of an anti-HER3 antibody treatment on HER3 activation and its association with p85 which leads to receptor internalization or degradation;

FIGS. 5B and 5C depict the effects of an anti-HER3 antibody (c1.105.5) on melanoma cell migration in vitro;

FIG. 6A depicts the effect of HER3 knock-down on the proliferation of Mel Gerlach melanoma cells;

FIG. 6B depicts the effect of HER3 knock-down on the AKT activity in Mel Gerlach melanoma cells;

FIG. 6C depicts HER3 knock-down sensitization of Mel Gerlach melanoma cells to Dacarbazine-induced apoptosis;

FIG. 7A depicts the effect of HER3 knock-down in Mel Juso melanoma cells;

FIG. 7B depicts HER3 knock-down inhibition of the proliferation of Mel Juso melanoma cells;

FIG. 7C depicts anti-HER3 monoclonal antibody (cl. 2D1D12) inhibition of the invasion of Mel Juso melanoma cells;

FIGS. 8A-C depict HER3 and HER2 surface expression measured by indirect flow cytometry; and

FIG. 9 depicts the effect of HER3 knock-down on apoptosis in melanoma cells.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

Embodiments described herein relate, in one aspect, to an inhibitor of HER3 for the treatment of melanoma, preferably melanoma metastases and/or refractory melanoma.

As used herein the term “inhibitor of HER3” includes every compound which may inhibit the transcription or translation of HER3 or which may act on the protein level, i.e. inhibit the HER3 activity, in particular HER3 mediated signal transduction. The activity of HER3 may also be inhibited on the gene level. Inhibition on the gene level may comprise a partial or complete gene inactivation, e.g. by gene disruption.

The terms “inhibitor of HER3” and “HER3 inhibitor” may be used interchangeable herein.

In an embodiment the HER3 inhibitor is selected from the group consisting of nucleic acids, in particular small interfering RNA (siRNA), antisense oligonucleotides or ribozymes, peptidic compounds, in particular antibodies or antibody fragments and small organic non-peptidic molecules, i.e. molecules having a low molecular weight, and combinations thereof. Preferably, the HER3 inhibitor according to the invention is an anti-HER3-antibody or HER3 specific siRNA.

As used herein, the term “antibody” covers monoclonal antibodies, polyclonal antibodies, multispecific antibodies (e.g. bispecific antibodies) formed from at least two antibodies and antibody fragments as long as they exhibit the desired activity.

An especially preferred anti-HER3 antibody is an antibody directed against the extracellular domain of HER3. Said anti-HER3 antibody is preferably monoclonal.

A monoclonal antibody may be obtained by the hybridoma method as described by Kohler et al. (Nature 256 (1975), 495) or by recombinant DNA methods (cf. e.g. U.S. Pat. No. 4,816,567). Monoclonal antibodies may also be isolated from phage antibody libraries using techniques which are known to the person skilled in the art. The antibody may be an IgM, IgG, e.g. IgG1, IgG2, IgG3 or IgG4.

Antibody fragments include a portion of an antibody, generally the antigen binding or variable region of the intact antibody. Examples of antibody fragments include Fab, Fab', F(ab')2 and Fv fragments, diabodies, single chain antibody molecules and multispecific antibody fragments.

Particularly, the antibody may be a recombinant antibody or antibody fragment, more particularly selected from chimeric antibodies or fragments thereof and diabodies. For therapeutic purposes, particularly for the treatment of humans, the administration of chimeric antibodies, humanized antibodies or human antibodies is especially preferred.

The anti-HER3 antibodies may be coupled to a labeling group, particularly for diagnostic applications. Examples for suitable labeling groups such as radioactive groups, fluorescent groups or other labeling groups are known in the art. Further, particularly for therapeutic applications, the antibody may be coupled to an effector group, which may be effective in the treatment of melanoma, e.g. a cytotoxic group such as a radioactive group, a toxin or another effector group as known in the art.

SiRNA as used herein is a double-strand of RNA and/or nucleotide analogues with 3′ overhangs on at least one end, preferably either ends. Each RNA strand of the double-strand has a 5′ phosphate group and a 3′ hydroxyl group. Preferably, each RNA strand of the double strand is 20 to 25 nucleotides long, more preferably 20 to 23 nucleotides and most preferably 21 nucleotides. The 3′ overhang on the end of a RNA strand is preferably 2 nucleotides long. In a particular preferred embodiment the siRNA double-strand consists of two 21 nucleotides long RNA strands each having a 2 nucleotides long 3′ overhang. Methods for obtaining siRNA molecules are known to the person skilled in the art.

SiRNA molecules may be applied to a target cell by any technique which is known to the person skilled in the art, such as transfection of exogenous siRNA or of an appropriate vector, e.g. viral or non-viral, producing a single transcript, which can be processed into a functional siRNA.

Ribozymes may be selected from RNA molecules and nucleic acid analogues. Suitable anti-sense molecules may be selected from DNA molecules, RNA molecules and nucleic acid analogues.

In an embodiment the HER3 inhibitor is an inhibitor of HER3 expression, which preferably acts by down-regulation or knock-down of HER3. Down-regulation or knock-down of HER3 results preferably in an at least partial disappearance of HER3 molecules from the cell surface. In a particular preferred embodiment HER3 expression is inhibited by the use of specific siRNAs. Especially preferred HER3 specific siRNAs are selected from the group consisting of:

5′ GGCUAUGUCCUCGUGGCCAtt 3′ (sense1);
5′ UGGCCACGAGGACAUAGCCtg 3′ (antisense1);
5′ GGCAGUGUGUCCUGGGACUtt 3′ (sense2);
5′ AGUCCCAGGACACACUGCCtg 3′ (antisense2);
or a combination thereof.

HER3 down-regulation or knock-down may lead to impaired AKT activation, reduced Cyclin B1 levels and/or decreased Rb phosphorylation. Preferably, HER2 protein levels are not altered in HER3 down-regulated cells.

In a particular preferred embodiment, HER3 down-regulation or knock-down inhibits melanoma cell proliferation, migration and/or invasion.

In a further preferred embodiment the HER3 inhibitor is an inhibitor of Heregulin-induced HER3 activation. The HER3 inhibitor may in particular inhibit Heregulin-induced HER3 phosphorylation leading to receptor internalization or degradation. The inhibitor may influence the binding of Heregulin to HER3, particularly by decreasing the binding of Heregulin to HER3. In a particular preferred embodiment, the HER3 inhibitor, preferably an anti-HER3 antibody, is used to inhibit Heregulin-induced migration and invasion of melanoma cells.

In yet a further embodiment the HER3 inhibitor is characterized in that binding of the inhibitor to HER3 reduces HER3 mediated signal transduction. HER3 mediated signal transduction is preferably reduced by a down-regulation of HER3 resulting in an at least partial disappearance of HER3 molecules from the cell surface or by a stabilization of HER3 on the cell surface in a substantially inactive form, i.e. a form which exhibits a lower signal transduction compared to the non-stabilized form. An anti-HER3-antibody is a preferred embodiment of a HER3 inhibitor reducing HER3 mediated signal transduction.

According to the invention the term “melanoma” denotes preferably malignant tumors of melanocytes which are found predominantly in skin but also in the bowel and the eye. Malignant melanoma is due to uncontrolled growth of pigment cells, i.e. melanocytes. According to an especially preferred embodiment, refractory melanoma is primary melanoma or metastases or therapy resistant melanoma. Metastases, in particular micrometastases, are often not accessible by surgery and need pharmacological or/and irradiation treatment. As used herein, the term “melanoma” includes “melanoma metastases”.

As used herein, the terms “refractory melanoma” or “therapy resistant melanoma” include the inability of hyperproliferative cells/cancer cells, in particular melanoma cells, to respond to therapy, i.e. the inability of a therapeutic treatment to reduce or suppress cell proliferation or/and to induce cell death in hyperproliferative cells/cancer cells. A serious form of therapy resistance included herein is multi-drug resistance. “Therapy resistant” or “refractory melanoma” also includes resistance of melanoma against a monotherapy, in particular with a cytostatic, cytotoxic or/and chemotherapeutic agent such as an apoptosis-inducing agent or by irradiation therapy. “Therapy resistant” or “refractory melanoma” also includes resistance to a combination treatment of at least two selected from cytostatic, cytotoxic and chemotherapeutic agents and irradiation. As used herein at least partially therapy resistant melanoma is also included. As used herein “at least partially therapy resistant” includes temporal development of therapy resistance from full responsiveness to an anti-cancer treatment regimen to complete resistance against this treatment.

The melanoma, in particular melanoma metastases, to be treated is preferably selected from the group consisting of malignant melanoma, clear cell sarcoma (melanoma of soft parts), mucosal melanoma and/or uveal melanoma or is derived therefrom. Common types of melanoma in the skin comprise superficial spreading melanoma (SSM), nodular melanoma, acral lentiginous melanoma and lentigo melanoma.

According to another embodiment the HER3 inhibitor used is an inhibitor of melanoma cell proliferation, migration and/or invasion, i.e. a reduction of HER3, in particular of HER3 expression or HER3 activity, results in reduced cell proliferation, migration and/or invasion. Tumor cell migration and invasion are important prerequisites for metastasis. Preferably, melanoma cell migration and/or invasion is blocked by inhibiting HER3 signaling via the PI3K pathway by a HER3 inhibitor, in particular an anti-HER3 antibody. In contrast, a monoclonal antibody specific for HER2 does not affect melanoma cell invasion. As already outlined above, according to another embodiment, melanoma cell proliferation, migration and/or invasion is inhibited by HER3 down-regulation or knock-down.

Preferably, the HER3 inhibitor is an enhancer of apoptosis. Moreover, the HER 3 inhibitor preferably sensitizes the melanoma for treatment with a further active agent or radiation therapy, i.e. acts synergistically with the active agent or radiation therapy. Such an active agent may be selected from the group consisting of chemotherapeutic agents, immunotherapeutic agents and/or adjuvant agents. Preferably, the agent is a chemotherapeutic agent. According to an especially preferred embodiment HER3 knock-down or down-regulation sensitizes melanoma cells to apoptosis.

A further aspect relates to a pharmaceutical composition including an inhibitor of HER3 as defined herein.

The pharmaceutical composition may be formulated by mixing the active agent, i.e. the HER3 inhibitor with physiologically acceptable carriers, diluents, excipients and/or adjuvants, e.g. in the form of lyophilized formulations, aqueous solutions, ointments, emulsions, suspensions, dispersions or solid preparations such as tablets, powders, dragees or capsules as described in Remington's Pharmaceutical Sciences.

The composition may be administered by injection, orally, topically, rectally, intranasally or by any other suitable means. The composition may be administered by one dose per day or may be divided up into several doses. The composition may be also administered in a continuous way, e.g. by infusion.

Furthermore, one may administer the pharmaceutical composition in a targeted drug delivery system, for example in a liposome coated with a tumor-specific antibody. The liposomes will be targeted to and taken up selectively by the tumor.

The effective amount of the active agent, i.e. the HER3 inhibitor in the composition may be determined by the skilled person without any undue burden depending on the kind of active agent and the kind of melanoma to be treated. For example, about 1 μg/kg to 15 mg/kg of a HER3 inhibitor, such as an anti-HER3 antibody, may be administered to a human patient, e.g. by one or more separate administrations or by continuous infusion. A typical daily dosage might range from about 1 μg/kg to about 100 mg/kg or more, depending on the factors such as age, gender, weight, tumor thickness and condition of the person to be treated etc. For repeated administrations over several days or longer, depending on the condition to be treated, the treatment is sustained until a desired suppression of disease symptoms occurs.

Radiation treatment regimens are known by a person skilled in the art.

In a preferred embodiment the pharmaceutical composition is to be administered in combination with further cytostatic, cytotoxic, chemotherapeutic and/or immunotherapeutic agents and/or before, during and/or after surgical removal of the melanoma and/or radiation therapy.

Thus, in a further preferred embodiment the pharmaceutical composition includes at least one further active agent, which is preferably active in the treatment of melanoma such as a further chemotherapeutic and/or immunotherapeutic agent.

Chemotherapeutic agents may preferably be selected from the group consisting of Dacarbazine (DTIC), Herceptin, Omnitarg, Lapatinib, Temozolomide and/or Procarbazine. Down-regulation of HER3 synergistically enhances Dacarbazine-induced apoptosis. Thus, Dacarbazine is an especially preferred chemotherapeutic agent.

Immunotherapeutic agents include for example interleukin-2 (IL-2) or interferon (IFN).

According to another embodiment, a pharmaceutical composition may be administered as part of a combination therapy. As used herein, “combination therapy” refers to the simultaneous administration of two or more active agents as defined above, wherein at least one of these agents is a HER3 inhibitor. The two or more active agents may be administered simultaneously in one single pharmaceutical composition or more than one pharmaceutical composition, wherein each composition includes at least one active agent. However, the term “combination therapy” refers also to other types of therapy such as radiation which are used at the same time. A preferred embodiment relates to a pharmaceutical composition for the treatment of patients expressing elevated levels of HER3.

As used herein, “elevated levels” of HER3 expression denote levels of HER3 expression in a patient showing melanoma or refractory melanoma, which are higher compared to a patient showing no melanoma.

It is demonstrated that HER3 is expressed in malignant melanoma and metastases at elevated levels. It was found that, high HER3 expression correlates with cell proliferation, tumor progression and/or reduced patient survival. HER3 is undetectable in primary melanocytes. Thus, HER3 expression may serve as a prognostic marker for melanoma.

Accordingly, one embodiment relates to the use of HER3 as prognostic marker for melanoma, in particular melanoma metastases.

The subject in need of treatment may be any animal which may suffer from melanoma, in particular a mammal, more particularly a human being.

A further aspect relates to an in vitro method for the diagnosis and/or prognosis of melanoma, in particular melanoma metastases, including
(a) detecting HER3 levels in cells to be analyzed and
(b) correlating the detected HER3 levels with proliferation, tumor progression and/or patient survival, and optionally
(c) administering a HER3 inhibitor or pharmaceutically composition.

Thus, according to an especially preferred embodiment, the method for the diagnosis and/or prognosis of melanoma, in particular melanoma metastases, may be combined with the administration of a pharmaceutical composition, especially if an elevated level of HER3 expression is detected.

In general, detection of HER3 levels can be done by any method which is known by the person skilled in the art. In particular, the HER3 levels or HER3 expression may be determined on the nucleic acid level or on the protein level according to standard methods, e.g. using a gene array or immunochemical, i.e. immunohistochemical methods. An overexpression of HER3 is determined by comparing the HER3 expression in the sample to be analysed with the HER3 expression in control samples, e.g. samples from healthy subjects or with standard values. The cells to be analyzed may be a tissue sample, e.g. a biopsy.

Commonly, up-regulated HER3 levels indicate melanoma.

The in vitro method may be combined with other methods for the diagnosis or prognosis of melanoma which are known in the art such as dermatoscopic exam, biopsy, X-rays, ultrasound, lactate dehydrogenase (LDH) testing and/or photoacoustic detection.

### FIGURE LEGENDS

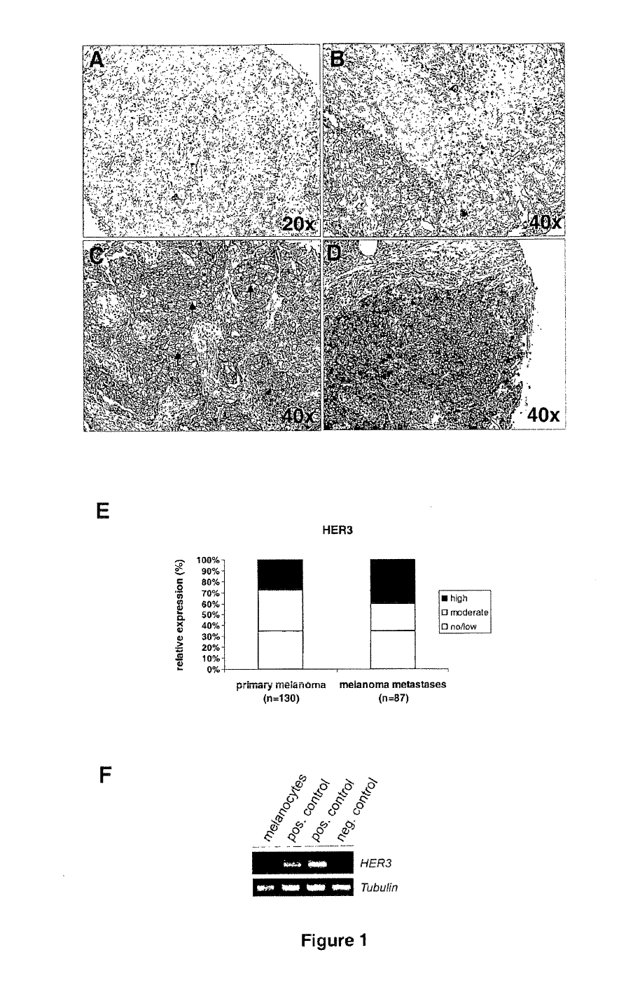

FIG. 1: HER3 expression in primary melanoma and metastases.

Immunohistochemical staining of HER3 in primary melanoma and metastases. A Low HER3 expression (20×), B Moderate HER3 expression (40×) and C High HER3 expression (40×) in primary melanoma. HER3 immunoreactivity is accentuated at the cell membrane (black arrows). D High HER3 expression in melanoma metastases (40×). E HER3 expression frequencies in primary melanoma and metastases. F Absence of HER3 expression in primary melanocytes. HER3 expressing and nonexpressing melanoma cell lines served as positive and negative controls, respectively. Tubulin served as a loading control.

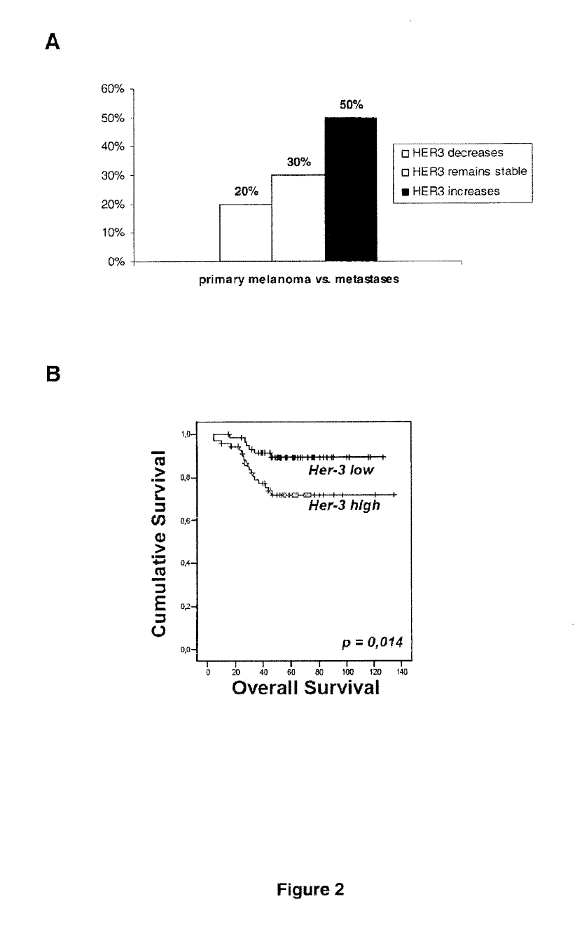

FIG. 2: HER3 protein expression confers poor prognosis in melanoma patients.

A HER3 expression increases during melanoma progression. 20 primary tumors with matching melanoma metastases where evaluated based on their GIS score for HER3. B Kaplan-Meier analysis of tumor-specific survival according to HER3 expression levels (p=0.014).

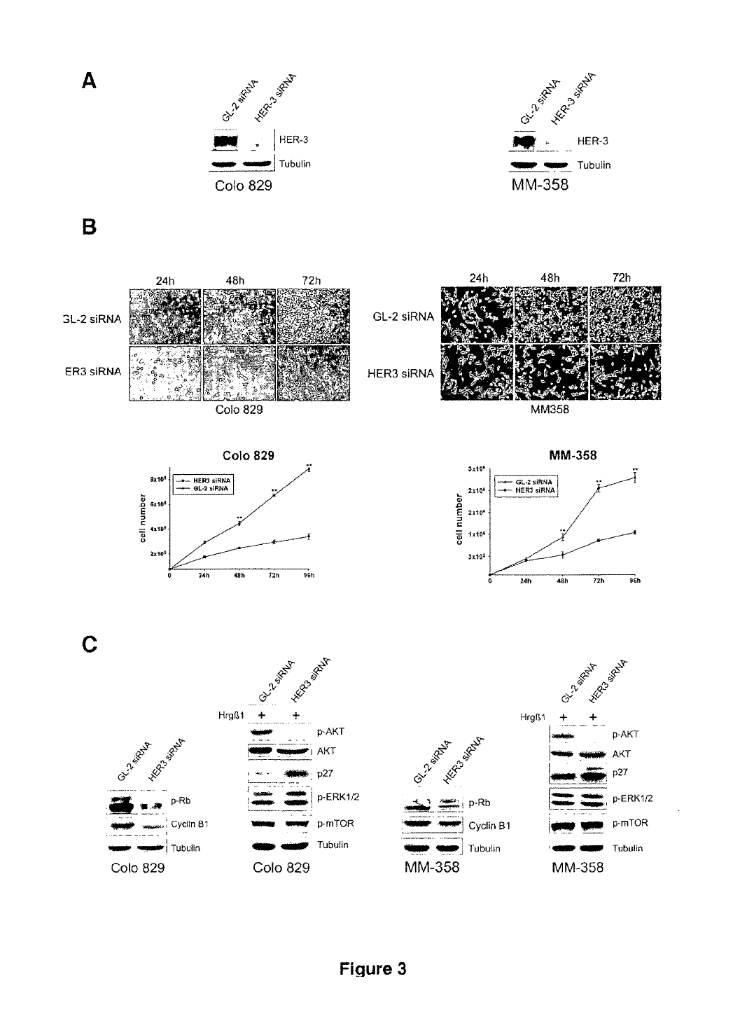

FIG. 3: HER3 knock-down inhibits melanoma cell proliferation

A HER3 knock-down in Colo 829 and MM-358 melanoma cells. Western blots are shown for HER3 and Tubulin. B HER3 knock-down inhibits proliferation of Colo 829 and MM-358 melanoma cells. Bright field pictures were taken using a Zwiss Axiovert 300 microscope at a 10× magnification and growth curves were done by counting cells at the indicated time points using an automated cell counter. The data are shown as mean±SDM. C HER3 knock-down impairs AKT activity upon heregulin β1 stimulation and induces a growth arrest in Colo 829 and MM-358 melanoma cells. Western blots are shown for, p-AKT, AKT, p27, p-Rb, Cyclin B1, p-ERK1/2, p-mTOR and Tubulin.

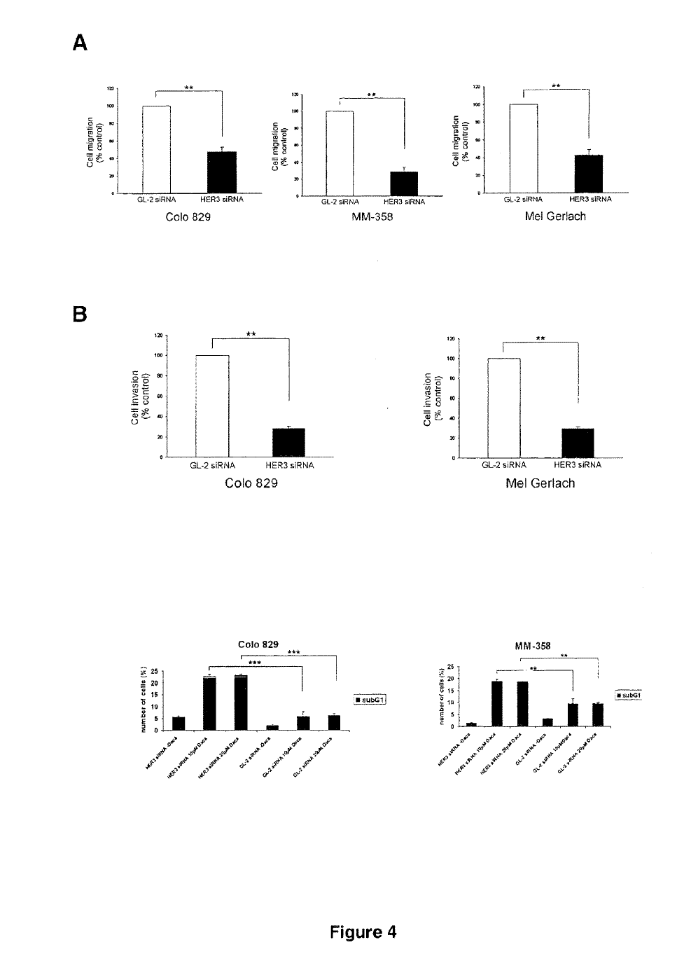

FIG. 4: HER3 knock-down inhibits melanoma cell migration and invasion and induces apoptosis in response to chemotherapeutic drugs.

A HER3 knock-down inhibits migration of Colo 829i, MM-358 and Mel Gerlach melanoma cells. For quantification, pictures of migrated cells were taken on a Zeiss Axiovert 300 microscope and cells were counted in at least 10 random fields. The data are shown as mean±SDM. B HER3 knock-down inhibits invasion of Colo 829 and Mel Gerlach melanoma cells. Quantification of invaded cells was done as described in A. C Induction of apoptosis in Colo 829 and MM-358 HER3 knock-down cells upon treatment with increasing amounts (10 μM and 20 μM) of Dacarbazine. Data are shown as mean±SDM.

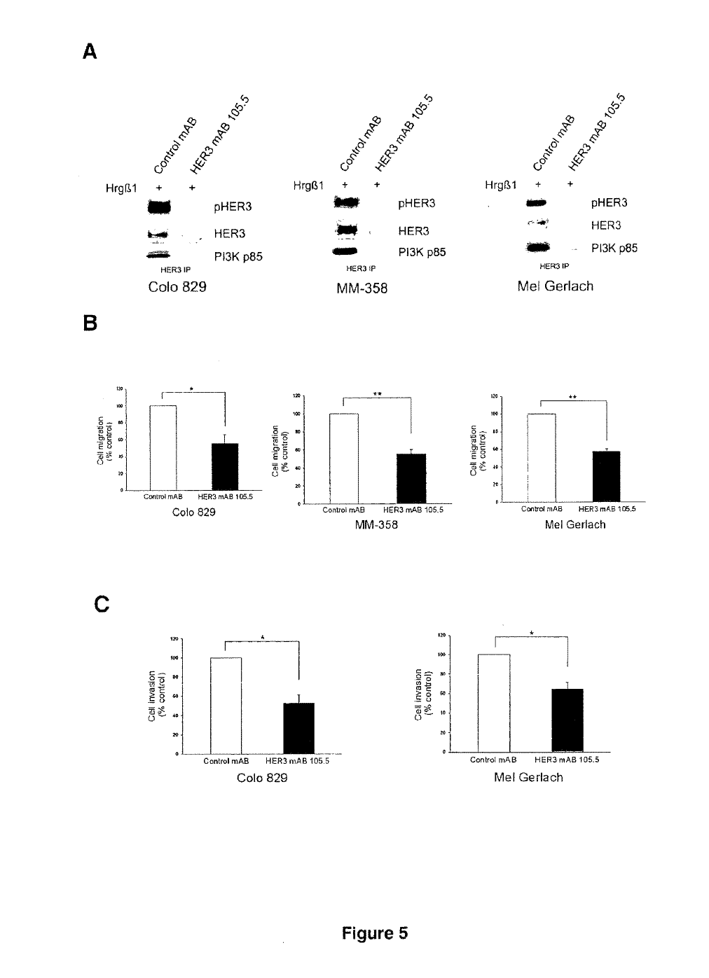

FIG. 5: An Anti-HER3 monoclonal antibody (cl. 105.5) inhibits HER3 activation and blocks melanoma cell migration and invasion.

A Anti-HER3 antibody treatment blocks HER3 activation, its association with p85 and leads to receptor internalization or degradation. Colo 829, MM358 and Mel Gerlach melanoma cells were either incubated with the HER3 blocking antibody (cl. 105.5) or an isotype control antibody, stimulated with Hrgβ1, lysed and equal amounts of protein was subjected to immunoprecipitations using a specific HER3 antibody. Western blots for p-HER3 (Y1289), HER3 and p85 are shown. B An Anti-HER3 antibody (c1.105.5) blocks melanoma cell migration in vitro. The cells were either incubated with the Anti-HER3 antibody or an isotype control antibody. The migration assay was done in a modified boyden chamber. Conditioned NIH3T3 medium containing 100 ng/ml Heregulin β1 was used as a chemoattractant. The quantification was done as described in FIG. 4. The data are shown as mean±SDM. C An Anti-HER3 antibody (cl. 105.5) blocks melanoma cell invasion in vitro. The assay was done as in B using growth factor-reduced matrigel in a modified boyden chamber.

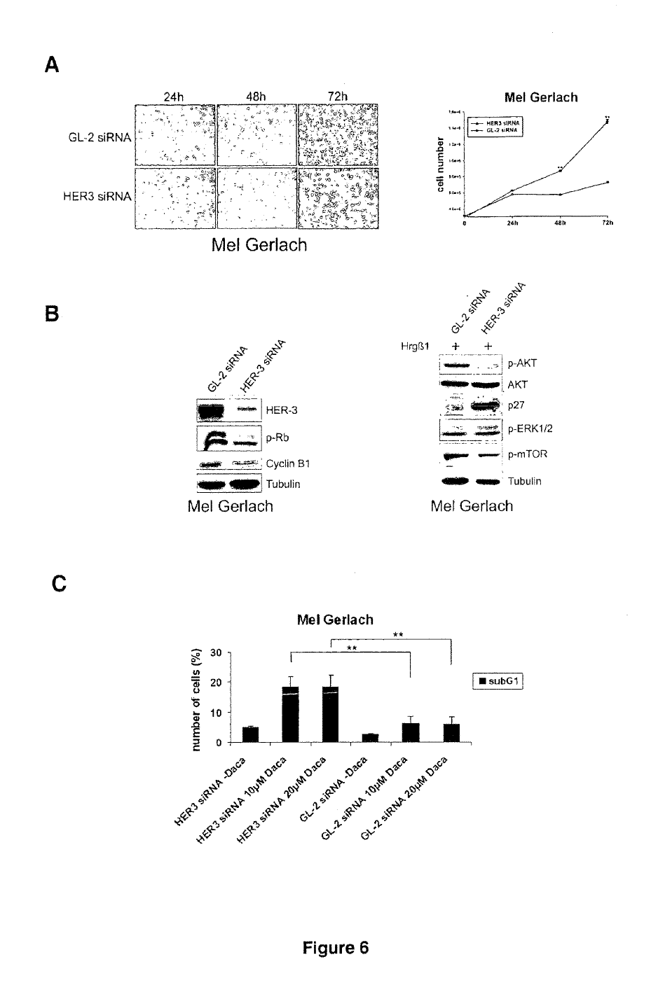

FIG. 6: HER3 knock-down in Mel Gerlach melanoma cells.

A HER3 knock-down inhibits proliferation of Mel Gerlach melanoma cells. HER3 knock-down and GL-2 control cells were counted at the indicated time points and data are shown as mean±SDM. Pictures were taken on a Zeiss Axiovert 300 microscope.

B. HER3 knock-down impairs AKT activity in Mel Gerlach melanoma cells. Western blots for HER3, p-AKT, AKT, p27, p-Rb, Cyclin B1, p-ERK1/2 and p-mTOR are shown. Tubulin served as loading control. C HER3 knock-down sensitizes Mel Gerlach melanoma cells to Dacarbazine-induced apoptosis. Mel Gerlach HER3 knock-down and GL-2 control cells were either treated with 10 or 20 μM Dacarbazine or left untreated for 48 hours and apoptosis was analyzed by flow cytometry.

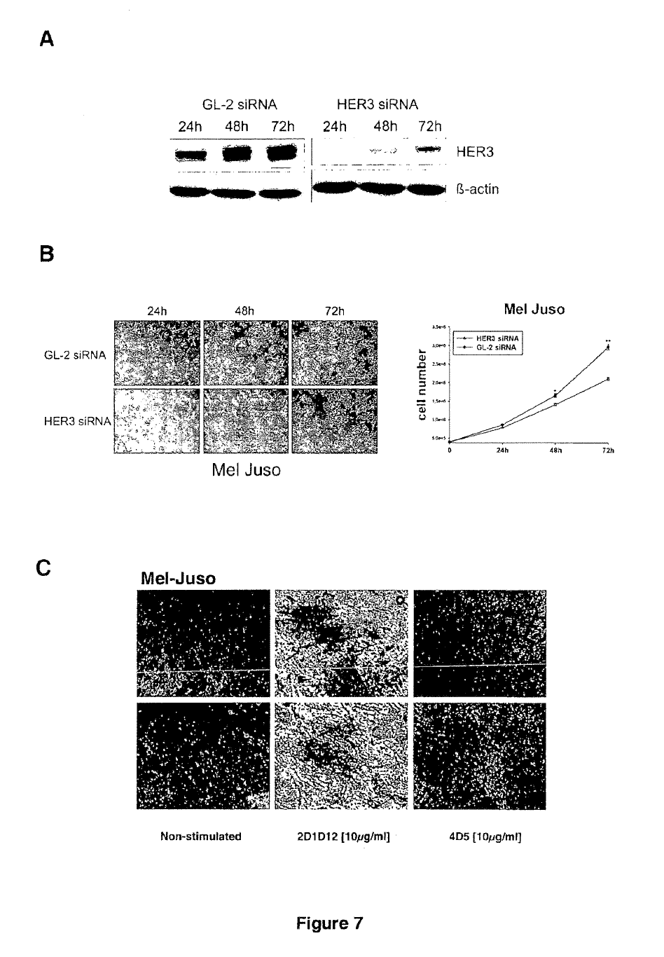

FIG. 7: HER3 knock-down in Mel Juso melanoma cells

A HER3 knock-down in Mel Juso melanoma cells. Cells were lysed at the indicated time points and subjected to Western blot analysis for HER3. β-actin served as a loading control. B HER3 knock-down inhibits proliferation of Mel Juso melanoma cells. C An Anti-HER3 monoclonal antibody (cl. 2D1D12) inhibits the invasion of Mel Juso melanoma cells. Mel Juso cells were either incubated with an anti-HER3 monoclonal antibody (cl. 2D1D12; 10 μf/ml), an anti-HER2 monoclonal antibody (4D5; 10 μg/ml) or left untreated.

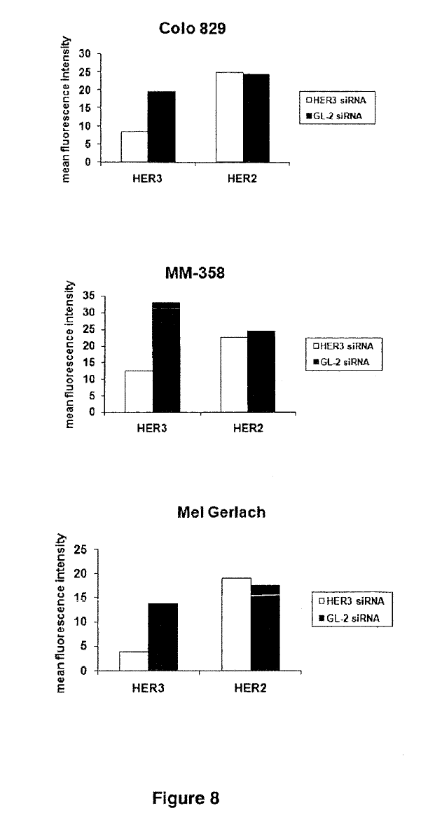

FIG. 8: HER3 knock-down does not alter HER2 surface expression in melanoma cell lines

A-C HER3 and HER2 surface expression was measured by indirect flow cytometry. Colo 829, MM-358 and Mel Gerlach HER3 knock-down cells were incubated with specific primary antibodies for HER2 and HER3 for 1 hour and afterwards with a PE-labelled secondary antibody followed by flow cytometry. The fluorescence intensities for HER3 and HER2 are shown.

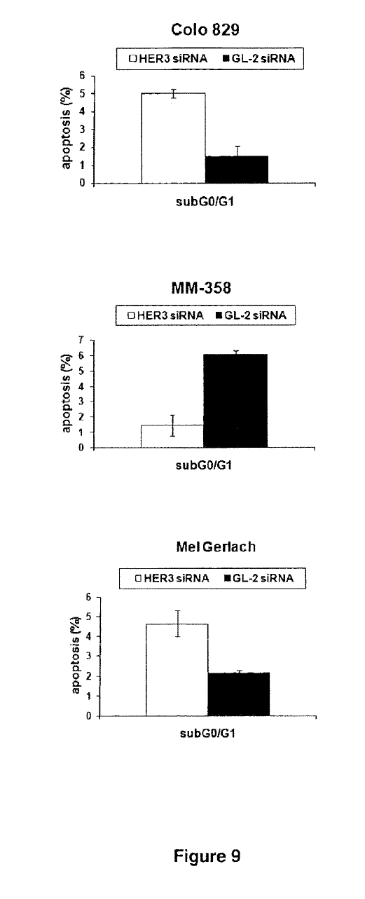

FIG. 9: HER3 knock-down does not induce apoptosis in melanoma cells

200,000 cells were seeded in 6 well plates and transfected with HER3 or GL-2 siRNAs using olgofectamine (Invitrogen). Cells were trypsinized after 48 hours and analyzed by Propidium Iodide staining as described in Material and Methods. Apoptotic cells were identified as the subG0/G1 population and quantified using the Cell Quest Pro software (Beckton Dickinson Biosciences).

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### 1. Material and Methods

### 1.1 Melanoma Patients

Formalin-fixed, paraffin embedded tissue of 130 primary cutaneous melanoma and 87 metastases was immunohistochemically analyzed for HER3. The patient age ranged from 19 to 90 years. Clinical follow-up was available in all of the patients (mean clinical follow up was 56±25 months). There were 60 nodular (NMM), 42 superficial spreading (SSM), 3 lentiginous (LMM), 9 acral lentiginous (ALM) and 16 not otherwise specified (NOS) melanoma. All melanoma had a Breslow tumor thickness between 0.4 and 17 mm. 53 of 130 patients (41%) had metastases during follow up and 24 of 130 patients (18%) died. Matched tumor samples of primary melanoma and metastases were available for 20 patients. 54 of the 130 patients with primary cutaneous melanomas were previously reported in a sentinel lymph node study (23). Approval was obtained from a local institutional Ethical Committee and written informed consent signed by all study participants.

### 1.2 Tissue Micro Array Construction and Immunohistochemistry

A morphologically representative region of a paraffin “donor” blocks was chosen to prepare the melanoma tissue micro arrays. The representative region was taken with a core tissue biopsy (diameter: 0.6 mm; height: 3-4 mm) and precisely arrayed into a new “recipient” paraffin block using a custom built instrument (Kononen J, Bubendorf L, Kallioniemi A, et al. Tissue microarrays for highthroughput molecular profiling of tumor specimens. Nat Med 1998; 4; 844-847). After the block construction was completed, 4.0 μm sections of the resulting tumor tissue micro array block were cut with a microtome and used for further analysis.

HER3 and Ki-67 immunohistochemistry was performed using a Ventana Benchmark automated staining system (Ventana Medical Systems, Tucson, Ariz.). For antigen retrieval, slides were heated with cell conditioner 1 (standard procedure). Endogenous biotin was blocked with the appropriate kit. Primary antibodies against HER3 (Santa Cruz, clone C-17, dilution 1:50) and Ki-67 proliferation antigen (clone MIB-1, dilution 1:20 were applied and revealed with the iVIEW DAB detection kit, yielding a brown reaction product. The signal was enhanced with the Ventana amplification kit. Slides were counterstained with hematoxylin prior to glass cover slipping. The specificity of the staining was controlled by using iso-type antibody controls, secondary antibody controls or blocking peptides. The analysis of the tissue micro arrays was done using a Zeiss Axiovert 300 microscope.

### 1.3 Evaluation of HER3 Expression

To determine the expression frequencies of HER3, a semi-quantitative scoring system was applied following the German Immunohistochemical scoring (GIS) system in which the final immuno-reactive score equaled the product of the percentage of positive cells times the average staining intensity. Percentage of positive cells was graded as follows: 0=negative, 1=up to 10% positive cells, 2=11 to 50%, 3=51 to 80%, 4=>80%. Staining intensity of 0=negative, 1=weakly positive, 2=moderately positive, 3=strongly positive. The extent of KI-67 staining was recorded as the Ki-67 Labelling Index (number of marked nuclei per 100 melanoma cells). All stainings were evaluated by two different investigators.

### 1.4 Statistical Analysis

The protein expression frequency for HER3 was analyzed by dividing the GIS score in three groups with GIS 1−4=no/low, GIS 5−8=moderate and GIS 9−12=high expression. For statistical analysis, two groups were considered which were divided through GIS-dichotomization at the median (low: GIS ≦6 and high: GIS >6). Correlations between HER3 and Ki-67 were analyzed using Spearman's rank correlation. The overall survival of melanoma patients was estimated by the Kaplan Meier method and differences between groups were assessed by the log-rank test. The overall survival was defined as the time of primary tumor diagnosis to the last follow-up visit or patient death. All p-values were calculated using the two-sided Fisher's exact Test or the paired Student's T-Test and p-values <0.05 were considered statistically significant. A multivariable Cox regression model was adjusted, testing the independent prognostic relevance of HER3 in melanoma patients. The following clinical variables were considered: age (≦60 years vs. >60 years), sex (male vs female), tumor thickness (≦2 mm vs >2 mm) and metastases during follow up. The proportionality assumption for all variables was assessed with log-negative-log survival distribution functions. For the analysis of matched tumor metastases pairs, the GIS values of HER3 in primary melanoma and melanoma metastases were directly compared and analyzed. The statistical analysis was performed with the SPSS 12.0 software (SPSS Inc., Chicago Ill.).

### 1.5 Cell Culture and Compounds

All cell lines used in this study were purchased from the American Type Culture Collection (ATCC) and cultured according to ATCC guidelines. Dacarbazine and Propidium-Iodide were purchased from SIGMA. Heregulin 131 was purchased from R&D Systems and diluted in PBS prior to use.

### 1.6 RNA Interference

HER3 siRNAs were obtained from AMBION. Two independent siRNAs were used in all experiments. Sequences for HER3 siRNAs were sense1 5′ GGCUAUGUCCUCGUGGCCAtt 3′, antisensel 5′ UGGCCACGAGGACAUAGCCtg 3′ and sense2 5′ GGCAGUGUGUCCUGGGACUtt 3′, antisense2 5′ AGUCCCAGGACACACUGCCtg 3′. The GL-2 siRNA (Dharmacon) was used as a negative control in all experiments. GL-2_sense 5′ CGUACGCGGAAUACUUCGAtt 3′, GL-2_antisense 5′ UCGAAGUAUUCCGCGUACGtt 3′. Transfection of siRNAs was done using Oligofectamine (Invitrogen, CA) according to manufacturer's recommendation.

### 1.7 Antibodies, RT-PCR and Western Blot Analysis

Antibodies against p-HER3 (Tyr 1289), p85, p-AKT (Ser473), CyclinB1, p-ERK1/2, pmTOR and p-Rb were all purchased from Cell Signaling (Beverly, Mass.). HRP conjugated rabbit secondary antibodies were from BioRad. Anti-Tubulin, Anti-13-actin and HRP-conjugated mouse secondary antibodies were from SIGMA. The anti-HER3 (clone 2F12) antibody was from Upstate and anti-HER3 (C-17) for immunohistochemistry as well as Akt 1/2 (H-136) were from Santa Cruz. Anti-p27 was purchased from Transduction Laboratories. Western blot analysis and immunoprecipitations were done as described previously (Hackel P O, Gishizky M, Ullrich A. Mig-6 is a negative regulator of the epidermal growth factor receptor signal. Biol Chem 2001; 382; 1649-1662.). Total RNA was isolated using the RNeasy Mini Kit (Quiagen) and c-DNA was synthesized using the AMV Reverse Transcriptase (Roche) according to manufacturer's recommendations. RT-PCR primers for HER3: HER3_fwd 5′CTCCGCCCTCAGCCTACCAGTT 3′ and HER3 rev 5′ TGCTCCGGCTTCTACACATTGACA 3′ (Tm=64° C.) and for Tubulin: Tubulin_fwd; 5′ AAGTGACAAGACCATTGGGGGAGG 3′ and Tubulin rev 5′ GGGCATAGTTATTGGCAGCATC 3′ (Tm=55° C.). All PCR reactions were done in an Eppendorf thermocycler (Eppendorf).

### 1.8 Proliferation Assay

75000 or 250000 cells were seeded in 24 well or 6 cm plates and transfected with HER3 or GL-2 siRNAs using oligofectamine (Invitrogen). The cells were grown in the presence of medium containing 10% FCS and counted (Coulter counter, Beckton Dickinson) at the indicated time points. The data are shown as mean±SDM.

### 1.9 Migration and Invasion Assay

200,000 cells were seeded in 6 well plates and transfected with HER3 or GL-2 siRNAs using oligofectamine (Invitrogen). The cells were serum-starved for 24 hours and 25.000 cells were either seeded on to a membrane or on to a growth factor reduced matrigel coated membrane with 8 μM pores of a modified boyden chamber (Schubert and Weiss) containing 500 μl serum-free medium. 10% fetal calf serum served as chemoattractant. The cells were allowed to migrate or invade for 20 or 24 hours, respectively. The cells were stained with crystal violet, washed in PBS and pictures were taken on a Zeiss Axiovert 300 microscope. For quantification cells in at least 10 random fields were counted and the data are shown as mean±SDM.

### 1.10 HER3 Blocking Antibody—Migration and Invasion Experiments

The HER3 blocking antibody (cl. 105.5) was purchased from Upstate, N.Y. The second HER3 blocking antibody (cl. 2D1D12) was generated in the Department of Molecular Biology at the Max-Planck Institute of Biochemistry.

The migration and invasion assays were performed as described previously (van der Horst E H, Murgia M, Treder M, Ullrich A. Anti-HER3 MAbs inhibit HER3-mediated signaling in breast cancer cell lines resistant to anti-HER2 antibodies. Int J Cancer 2005; 115; 519-527; and Albini A, Iwamoto Y, Kleinman H K, Martin G R, Aaronson S A, Kozlowski J M, McEwan R N. A rapid in vitro assay for quantitating the invasive potential of tumor cells. Cancer Res. 1987 Jun. 15; 47(12):3239-45). Briefly, 300.000 cells were seeded in 6 cm plates and serum-starved in medium containing 0.1% FCS for 24 hours. 200.000 cells per ml were incubated with 10 μg/m1 HER3 blocking antibody for 1 hour and 50.000 cells were then seeded either on to a membrane or on to a growth factor reduced matrigel coated membrane with 8 μM pores of a modified boyden chamber (Schubert and Weiss) containing 500 μl serum free medium. Conditioned NIH 3T3 medium containing 0.01% ascorbic acid and Heregulin β1 (100 ng/ml) was used as a chemoattractant. Migrated or invaded cells were stained by crystal violet, washed in PBS and analyzed using a Zeiss Axiovert 300 microscope. For quantification at least 10 random fields were counted and the data are presented as mean±SDM.

To asses the HER3 phosphorylation state, cells were serum-starved for 24 hours, incubated with 10 μg/ml blocking antibody for 1 hour, stimulated with 50 ng/ml Heregulin β1 for 2 hours, lysed and subjected to Immunoprecipitations using a specific HER3 antibody.

### 1.11 Apoptosis Assay (Propidium Iodide Staining)

200,000 cells were seeded into 6 well plates (Nunc) and transfected with HER3 or GL-2 siRNAs. Apoptosis was induced by adding either 10 or 20 μM Dacarbazine in DMSO to the medium. After 48 hours the supernatant of each reaction was collected and the cells were trypsinized. After centrifugation the cells were incubated for two hours in a Propidium-Iodide buffer (0.1% Na-Citrate, 0.1% Triton X-100, 20 μM Propidium-Iodide) and thereafter subjected to flow cytometric analysis (Beckton-Dickinson Biosciences). Apoptotic cells were identified as the sub G0/G1 peak and quantified using the Cell Quest Pro software (Beckton Dickinson).

### 1.12 Indirect Flow Cytometry

The antibodies used for HER3 and HER2 were described elsewhere (van der Horst E H, Weber I, Ullrich A. Tyrosine phosphorylation of PYK2 mediates heregulin-induced glioma invasion: novel heregulin/HER3-stimulated signaling pathway in glioma. Int J Cancer 2005; 113; 689-698; and van der Horst E H, Murgia M, Treder M, Ullrich A. Anti-HER3 MAbs inhibit HER3-mediated signaling in breast cancer cell lines resistant to anti-HER2 antibodies. Int J Cancer 2005; 115; 519-527). 500.000 cells were seeded in 10 cm dishes (Falcon) and transfected with HER3 or GL-2 siRNAs using oligofectamine (Invitrogen). Cells were collected after 24 hours using 10 mM EDTA and dissolved in 1 ml 3% FCS in PBS. The cell number was adjusted to 250.000 cells per reaction and cells were incubated for 30 minutes with 10 μg/ml of each primary antibody at 4° C. The cells were washed 3 times in 3% FCS/PBS and incubated with a PE-labeled secondary (1:1000) antibody at 4° C. for 30 minutes. After three more washes in 3% FCS/PBS the fluorescence intensity was measured in a flow cytometer (Beckton Dickinson Biosciences) and analyzed using the Cell Quest Pro software.

### 2. Results

### 2.1 HER3 is Frequently Expressed in Primary Melanoma and Metastases

HER3 protein expression was investigated in 130 primary malignant melanoma and 87 metastases using tissue micro arrays. HER3 immuno reactivity was accentuated at the cell membrane. In primary melanoma, moderate to high HER3 expression levels were found in 85 of 130 cases (65%) (FIGS. 1A-C and 1E). Furthermore, HER3 was highly expressed in 35 of 87 melanoma metastases (40%) (FIGS. 1D and 1E). Importantly, HER3 expression was undetectable in primary melanocytes (FIG. 1F). Interestingly, moderate to high HER3 expression significantly correlated with increased tumor cell proliferation (Ki-67 Labeling Index) in primary malignant melanoma (p=0.008; data not shown).

### 2.2 HER3 Expression in Melanoma Progression

Matched tumor samples of primary melanoma and metastases were studied in 20 patients. Remarkably, 10 of 20 (50%) patients showed an increase of HER3 expression in the metastases compared to the primary tumor. Six of 20 (30%) matched tumor samples showed a similar expression of HER3 and only 4 of 20 (20%) patients showed less HER3 expression in the metastases when compared to the primary tumor (FIG. 2A). These results show that in a majority of cases HER3 expression remains either stable or even increases during disease progression.

Importantly, Kaplan-Meier analysis showed that HER3 expression was significantly associated with tumor specific survival (p=0.014) (FIG. 2B). In a multivariate analysis, an adjusted Cox regression model was developed for the assessment of the overall survival rate. The characteristics of the variables are shown in Table 1.

TABLE 1
Multivariate analysis of factors possibly influencing
overall survival (forward LR method)

| Variable | Characteristics | Hazard (95% Cl) | ratio p |
|---|---|---|---|
| Age | 0 ≦ 60 years | — | NS |
| | 1 > 60 years | | |
| Sex | 0 = male | — | NS |
| | 1 = female | | |
| Tumor thickness | 0 ≦ 2 mm | — | NS |
| | 1 > 2 mm | | |
| HER-3 | 0 low | 2.6 (1.04-6.6) | 0.041^{a} |
| | 1 high | | |
| Metastases during | 0 no metastases | 22.2 (5.2-95.5) | 0.000 |
| follow-up | 1 metastases | | |

^{a}Bold type representing data with p < 0.05
Abbreviations: NS, not significant; HR, hazard ratio; 95% Cl, 95% confidence interval

The clinical variables used in the analysis were age, sex, metastases during progression, tumor thickness and HER3 expression (HER3 Immunohistochemistry score). In this model, metastases (p=0.000) and HER3 expression (p=0.041) were correlated with poor prognosis. the hazard ratio for death from melanoma concerning HER3 status was 2.6 (95% Confidence Interval: 1,042-6,671); accordingly, in cases with high HER3 expression, the probability of tumor-related death was almost three times higher than in cases with low HER3 staining. Because of the assumption of proportional hazards, the probability of melanoma-related death was consistently valid during the entire observation period. Taken together, these results indicate that HER3 is a critical parameter for melanoma prognosis and progression.

### 2.3 HER3 Knock-Down Inhibits Melanoma Cell Proliferation

In order to further characterize the role of HER3 in melanoma, HER3 was down-regulated by specific siRNAs in Colo 829, MM-358, Mel Gerlach and Mel Juso human melanoma cell lines (FIG. 3A, FIGS. 6A and 7A). Strikingly, depletion of HER3 strongly inhibited proliferation in these cell lines (FIGS. 3B, 6A and 7B). Importantly, HER3 predominantly signals via the PI3K-AKT pathway in the regulation of cell proliferation and -survival. Indeed, AKT activation was impaired in heregulin β1 stimulated knock-down cell lines indicating that HER3 signals via the PI3K-AKT pathway in melanoma cells (FIG. 3C and FIG. 6B). In contrast, p-ERK and pmTOR levels remained unchanged (FIG. 3C and FIG. 6B). In addition, an up-regulation of p27 protein levels was observed which is most likely a direct consequence of the impaired AKT activity (FIG. 3C and FIG. 6B) since AKT is known to trigger p27 degradation and cell cycle progression (30-33). To further characterize the mechanism of the growth inhibition, proteins implicated in cell cycle control were analyzed. As shown in FIG. 3C, HER3 knock-down led to reduced Cyclin B1 levels and decreased Rb phosphorylation. Importantly, HER2 protein levels were not altered in HER3 down-regulated cells suggesting that the effects are specific for HER3 (FIG. 8). The obtained data demonstrate that HER3 signals via the PI3K-AKTp27 pathway in melanoma cells and thereby appears to promote melanoma cell proliferation.

### 2.4 HER3 Knock-Down Inhibits Melanoma Cell Migration and Invasion

Melanoma metastases frequently express high levels of HER3 (FIGS. 1D and 1E). Given the association between HER3 expression and poor survival, one might hypothesize that HER3 plays a role in melanoma progression. Increased tumor cell migration and invasion are important prerequisites for metastasis. In order to address this question, the migration and invasion of Colo 829, Mel Gerlach and MM-358 melanoma cells upon interference with HER3 expression were analyzed. As shown in FIG. 4A, HER3 knock-down efficiently blocked melanoma cell migration in all three cell lines. In invasion experiments Colo 829 and Mel Gerlach cells were markedly inhibited upon HER3 suppression (FIG. 4B) while MM-358 cells did not invade the matrix even in untransfected controls (data not shown). These results establish HER3 as a potent mediator of melanoma cell migration and invasion.

### 2.5 HER3 Knock-Down Sensitizes Melanoma Cells to Dacarbazine-Induced Apoptosis

In contrast to previously published data in lung cancer cells which undergo apoptosis in the absence of HER3, suppression of HER3 and subsequent down-regulation of AKT activity did not induce apoptosis in melanoma cells (FIG. 9). Nevertheless, it reasoned that inhibition of HER3 might synergize with chemotherapy in the induction of apoptosis in melanoma cells. To date, Dacarbazine is the only FDA-approved drug for melanoma therapy. Indeed, it was found that Dacarbazine-induced apoptosis was significantly increased in HER3 knock-down melanoma cells (FIG. 4C and FIG. 6C). These results indicate that combination therapy with HER3 and Dacarbazine-like drugs might be useful for the treatment of malignant melanoma.

2.6 Anti-HER3 Monoclonal Antibodies Block Heregulin-Induced her3 Activation and Melanoma Cell Migration and Invasion

It has been shown above that HER3 is frequently overexpressed in primary melanoma and melanoma metastases and that high HER3 levels confer poor prognosis for melanoma patients. In addition, the RNA interference experiments shown above suggest that HER3 may be a potential target for melanoma therapy. To test this hypothesis in vitro Colo 829, MM-358, Mel Gerlach and Mel Juso melanoma cells were treated with anti-HER3 monoclonal antibodies. Remarkably, Heregulin induced activation of HER3 and its association with the PI3-K subunit p85 was completely abrogated in antibody-treated cells when compared to controls (FIG. 5A). In addition, anti-HER3 monoclonal antibodies cause receptor degradation or internalization similar to previously obtained results in breast cancer cells. Importantly, Anti-HER3 monoclonal antibodies are able to block Heregulin-induced migration and invasion of human melanoma cell lines (FIGS. 5B, 5C and FIG. 7C) indicating that such antibodies may be effective anti-melanoma therapeutics. Taken together, these results indicate that targeting HER3 is a promising new opportunity for melanoma therapy.

### 3. Discussion

According to the present invention, HER3 expression in melanoma and its significant association with proliferation are shown. Furthermore, frequent and high HER3 expression in melanoma metastases compared to primary melanoma is demonstrated, indicating that HER3 may be involved in disease progression. In addition, high levels of HER3 significantly correlated with decreased life expectancy of patients establishing HER3 as a novel prognostic marker for melanoma. Importantly, HER3 expression is undetectable in primary melanocytes suggesting that HER3 overexpression specifically occurs during melanoma development. To address the role of HER3 in melanoma development and progression, human melanoma cell lines upon siRNA interference with HER3 expression were analyzed. Reduction of HER3 expression resulted in reduced cell proliferation, migration and invasion. On the molecular signaling level, suppression of HER3 led to reduced AKT activity and increased p27 protein levels which may be the cause of the observed growth inhibition. Notably, HER3 ablation did not affect the ERK1/2 and mTOR kinases suggesting that inhibition of other downstream pathways such as PI3K-AKT seems to be sufficient to block melanoma cell proliferation, migration and invasion. To further analyze whether HER3 may qualify as a novel target in melanoma therapy, human melanoma cells lines were treated with anti-HER3 monoclonal antibodies. It was found that such antibodies can inhibit heregulin-induced HER3 phosphorylation leading to receptor internalization or degradation. Furthermore, it is shown that binding of p85, the regulatory subunit of PI3K, to HER3 is abrogated upon antibody incubation demonstrating that signaling via the PI3K/AKT signaling pathway is inhibited in these cells. Importantly, melanoma cell migration and invasion are greatly reduced in antibody-treated cells when compared to controls. These data demonstrate that anti-HER3 antibodies can inhibit HER3 signaling via the PI3K pathway in melanoma cell lines and thereby seem to block melanoma cell migration and invasion. Importantly, it was shown in Mel Juso melanoma cells that a monoclonal antibody specific for HER2 did not affect melanoma cell invasion suggesting that inhibition of HER3 alone is sufficient to block melanoma invasiveness (FIG. 7C).

It was shown that the PI3K-AKT signaling pathway has essential functions in the regulation of cell survival and it is often found up-regulated in human cancer cells. Although interference with HER3 function and the consequential down-regulation of AKT were not sufficient to induce major apoptosis of melanoma cells, it was tested whether a combination of HER3 down-regulation with chemotherapeutic drug treatment would increase cell death. HER3 knock-down cells were highly sensitive to apoptosis induced by the FDA-approved drug Dacarbazine suggesting that a combination with agents interfering with HER3 might prove effective in the treatment of malignant melanoma.

Taken together, our results establish HER3 as a target for melanoma therapy.

In this patent, certain U.S. patents, U.S. patent applications, and/or other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments.

Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. Inhibitor of HER3 for the treatment of melanoma metastases and/or refractory melanoma.

2. The inhibitor of HER3 according to claim 1, wherein the inhibitor of HER3 is an inhibitor of HER3 expression, which acts by down-regulation of HER3.

3. The inhibitor of HER3 according to claim 1, wherein the inhibitor of HER3 is an inhibitor of Heregulin-induced HER3 activation.

4. The inhibitor of HER3 according to claim 1, wherein the inhibitor of HER3 is an inhibitor of melanoma cell proliferation, migration and/or invasion.

5. The inhibitor of HER3 according to claim 1, wherein the inhibitor is selected from the group consisting of nucleic acids, antisense oligonucleotides or ribozymes, peptidic compounds, small organic molecules and combinations thereof.

6. The inhibitor of HER3 according claim 1, wherein the refractory melanoma is primary melanoma or metastases.

7. The inhibitor of HER3 according to claim 1, wherein the melanoma metastases are derived from or the refractory melanoma is selected from the group consisting of malignant melanoma, clear cell sarcoma, mucosal melanoma and/or uveal melanoma.

8. The inhibitor of HER3 according to claim 1, wherein the inhibitor of HER3 is an enhancer of apoptosis.

9. The inhibitor of HER3 according to claim 1, wherein the inhibitor of HER3 sensitizes the melanoma metastases and/or the refractory melanoma for treatment with a further agent, preferably a chemotherapeutic agent or radiation therapy.

10. Pharmaceutical composition comprising an inhibitor of HER3 for the treatment of melanoma metastases and/or refractory melanoma.

11. The pharmaceutical composition according to claim 10, further comprising one or more chemotherapeutic and/or immunotherapeutic agents.

12. The pharmaceutical composition according to claim 11, wherein the further agent is Dacarbazine, Herceptin, Omnitarg, Lapatinib and/or Temozolomide.

13-14. (canceled)

15. In vitro method for the diagnosis and/or prognosis of melanoma, comprising
(a) detecting HER3 levels in cells to be analyzed and
(b) correlating the detected HER3 levels with proliferation, tumor progression and/or patient survival and optionally
(c) administering an inhibitor of HER3 or a pharmaceutical composition comprising an inhibitor of HER3.

16. The in vitro method according to claim 15, wherein up-regulated HER3 levels indicate melanoma.

17. The inhibitor of HER3 according to claim 1, wherein the inhibitor is an siRNA.

18. The inhibitor of HER3 according to claim 1, wherein the inhibitor is an antibody or an antibody fragment.

19. A method of treating melanoma metastases and/or refractory melanoma comprising administering an effective amount of an inhibitor of HER3.

20. The method of claim 19, further comprising administering one or more chemotherapeutic and/or immunotherapeutic agents in combination with the inhibitor of HER 3.

21. The method of claim 20, wherein the HER3 inhibitor and/or the chemotherapeutic and/or immunotherapeutic agents are administered before, during and/or after surgical removal of the melanoma and/or radiation therapy.


SEQUENCE LISTING (10 sequences)

SEQ ID NO: 1 - DNA, 21 bp, Artificial
  HER3 siRNA sense 1
ggcuauguccucguggccatt

SEQ ID NO: 2 - DNA, 21 bp, Artificial
  HER3 siRNA antisense 1
uggccacgaggacauagcctg

SEQ ID NO: 3 - DNA, 21 bp, Artificial
  HER3 siRNA sense 2
ggcaguguguccugggacutt

SEQ ID NO: 4 - DNA, 21 bp, Artificial
  HER3 siRNA antisense 2
agucccaggacacacugcctg

SEQ ID NO: 5 - DNA, 21 bp, Artificial
  GL-2 siRNA sense
cguacgcggaauacuucgatt

SEQ ID NO: 6 - DNA, 21 bp, Artificial
  GL-2 siRNA antisense
ucgaaguauuccgcguacgtt

SEQ ID NO: 7 - DNA, 22 bp, Artificial
  HER3 primer_fwd
ctccgccctcagcctaccagtt

SEQ ID NO: 8 - DNA, 24 bp, Artificial
  HER3 primer_rev
tgctccggcttctacacattgaca

SEQ ID NO: 9 - DNA, 24 bp, Artificial
  Tubulin primer_fwd
aagtgacaagaccattgggggagg

SEQ ID NO: 10 - DNA, 22 bp, Artificial
  Tubulin primer_rev
gggcatagttattggcagcatc